(19) United States
(12) Patent Application Publication
(10) Pub. No.: US 2026/0191643 A1
(43) Pub. Date: Jul. 9, 2026

(54) BALLOON-EXPANDABLE PEDIATRIC SURGICAL HEART VALVE

(71) Applicant: Edwards Lifesciences Corporation, Irvine, CA (US)

(72) Inventors: Rodolfo Rodriguez, San Luis Obispo, CA (US); Milton Deherrera, Irvine, CA (US); Van Huynh, Anaheim, CA (US)

(21) Appl. No.: 19/560,612

(22) Filed: Mar. 9, 2026

Related U.S. Application Data
(63) Continuation of application No. PCT/US2024/046549, filed on Sep. 13, 2024.
(60) Provisional application No. 63/582,807, filed on Sep. 14, 2023.

(51) Int. Cl. A61F 2/24

(52) U.S. Cl. CPC A61F 2/2418; A61F 2220/0075; A61F 2230/0004; A61F 2230/0017; A61F 2250/0069; A61F 2250/0082

(57) ABSTRACT

A prosthetic surgical heart valve for smaller patients, in particular pediatric patients. The heart valves are smaller and more flexible, and may be incrementally expandable to accommodate growth in the patient. The heart valves are especially useful at the pulmonary valve position, where the opening and closing pressures are much lower than at the other native valve locations. The heart valves are formed of thinner and more flexible materials than aortic or mitral valves which enable them to open and close more effectively when subjected to the lower pulmonary blood flow pressures. Incremental expansion of the heart valves is enabled by a plastically-expandable stiffening band coupled to a valve member.

### CROSS REFERENCE TO RELATED APPLICATIONS

This application is a continuation of International Patent Application No. PCT/US2024/46549, filed September 13, 2024, which claims the benefit of U.S. Patent Application No. 63/582,807, filed September 14, 2023, the entire contents all of which are incorporated by reference for all purposes.

### TECHNICAL FIELD

The present application relates generally to expandable surgical pulmonic valves for adolescents, children and infants.

### BACKGROUND

In vertebrate animals, the heart is a hollow muscular organ having four pumping chambers: the left and right atria and the left and right ventricles, each provided with its own one-way valve. The natural heart valves are identified as the aortic, mitral (or bicuspid), tricuspid and pulmonary, and each has flexible leaflets extending inward from a fibrous annulus that coapt against each other to prevent reverse flow.

Prostheses exist to correct problems associated with impaired heart valves. For example, mechanical and tissue-based heart valve prostheses can be used to replace impaired native heart valves. Substantial effort has been dedicated to developing replacement heart valves, particularly tissue-based replacement heart valves. Recently, pediatric heart valve replacement surgery has become more common, often for the pulmonary (or pulmonic) valve located between the right ventricle and the pulmonary artery. Pediatric pulmonary valve problems may occur on their own or with other heart conditions present at birth (congenital heart defects), such as tetralogy of Fallot.

However, as most heart valve replacements are needed for late-stage heart disease in older patients, in particular for the mitral and aortic valves, the most common valve implants and technical advancements are too large and not well-suited to pediatric patients with smaller annulus or vessel diameters. For example, a retrospective review of all patients who underwent pulmonary valve replacement with a stented bioprosthesis from 1992 to 2008 was reported in “Bioprosthetic pulmonary valve replacement: Contemporary analysis of a large, single-center series of 170 cases,” Chen, et al., Volume 146, Issue 6, 2013, Journal of Thoracic and Cardiovascular Surgery. One conclusion reached was that the common technique of oversizing a stented bioprosthesis in a smaller, younger patient would not be beneficial. In younger patients, even the smallest available stented bioprosthetic valves (19 mm) will be oversize, because a 19-mm pulmonary valve is the normal valve diameter for a patient with a body surface area greater than 1 m², which is about average for a 10-yr old.

Consequently, there exists a need for a better understanding of the technical challenges in pediatric heart valve replacement surgery, and better valves therefor.

### SUMMARY

The present application provides exemplary prosthetic heart valves designed for smaller patients, in particular pediatric patients. The prosthetic heart valves are made smaller and more flexible, and may be incrementally expandable to accommodate growth in the patient. The heart valves are especially useful at the pulmonary valve position, where the opening and closing pressures are much lower than at the other native valve locations. As such, the heart valves are formed of thinner and more flexible materials which enable them to open and close more effectively when subjected to the lower pulmonary blood flow pressures.

A first embodiment of a prosthetic heart valve adapted for surgical delivery and implant at a native annulus comprises a valve member with a leaflet support structure defining a plurality of upstanding commissures alternating with the same number of arcuate cusps to define an undulating outflow edge. The valve member has a plurality of flexible leaflets extending along and attached to the undulating outflow edge that coapt against one another and form a one-way valve across an inner flow orifice defining a central axis. The support structure is formed of a plastically expandable material. A stiffening band juxtaposed against an inflow end of the valve member defines a generally annular shape with an initial diameter sufficient for valve functioning. The stiffening band is formed of a plastically expandable material, is incapable of compression smaller than the initial diameter, but is capable of expansion of at least 2 mm in diameter. Lastly, fabric coverings are provided around the valve member and stiffening band, wherein fabric coverings and/or sutures connecting the fabric covering to the valve member and stiffening band supply the only coupling structure between the valve member and stiffening band and there are no other non-fabric components therebetween.

A second exemplary prosthetic heart valve adapted for surgical delivery and implant at a native annulus has a valve member with a leaflet support structure defining a plurality of upstanding commissures alternating with the same number of arcuate cusps to define an undulating outflow edge. The valve member again has a plurality of flexible leaflets extending along and attached to the undulating outflow edge that coapt against one another and form a one-way valve across an inner flow orifice defining a central axis. The support structure is formed of a plastically expandable material, and the leaflets each have a thickness t of between about 0.004–0.013 inches (0.1–0.33 mm). A stiffening band juxtaposed against and coupled to an inflow end of the valve mem member defines a generally annular shape with an initial diameter sufficient for valve functioning. The stiffening band is formed of a plastically expandable material, is incapable of compression smaller than the initial diameter, but is capable of expansion of at least 2 mm in diameter. Finally, fabric coverings are provided around the valve member and stiffening band.

A still further exemplary prosthetic heart valve adapted for surgical delivery and implant at a native annulus includes a valve member with a leaflet support wireform defining a plurality of upstanding commissures alternating with the same number of arcuate cusps to define an undulating outflow edge. The wireform has a wire diameter d of between about 0.009‒0.019 inches (about 0.23‒0.48 mm), the valve member having a plurality of flexible leaflets extending along and attached to the undulating outflow edge that coapt against one another and form a one-way valve across an inner flow orifice defining a central axis. The wireform is formed of an expandable material. A stiffening band juxtaposed against and coupled to an inflow end of the valve member defines a generally annular shape with an initial diameter sufficient for valve functioning. The stiffening band is formed of a plastically expandable material, is incapable of compression smaller than the initial diameter, but is capable of expansion of at least 2 mm in diameter. Again, fabric coverings are provided around the valve member and stiffening band.

A method of surgically implanting a prosthetic heart valve at a native pulmonary annulus and enlarging the size of the implanted prosthetic heart valve disclosed herein includes the steps of:

providing a prosthetic heart valve having:

a valve member with a leaflet support structure defining a plurality of upstanding commissures alternating with the same number of arcuate cusps to define an undulating outflow edge, the valve member having a plurality of flexible leaflets extending along and attached to the undulating outflow edge that coapt against one another and form a one-way valve across an inner flow orifice defining a central axis, the support structure being formed of a plastically expandable material;

a stiffening band juxtaposed against an inflow end of the valve member, the stiffening band defining a generally annular shape with an initial diameter sufficient for valve functioning and being formed of a plastically expandable material and being incapable of compression smaller than the initial diameter, the stiffening band being capable of expansion of at least 2 mm in diameter; and

fabric coverings around the valve member and stiffening band, wherein the fabric coverings or sutures connecting the fabric covering supply the only coupling structure between the valve member and stiffening band;

surgically delivering and implanting the prosthetic heart valve at the native pulmonary annulus by expanding the prosthetic heart valve;

closing up the patient after implanting the prosthetic heart valve at the native pulmonary annulus;

periodically measuring a size of the native pulmonary annulus or an analog of the size; and

when the size or analog reaches a threshold magnitude, advancing an expansion balloon within the prosthetic heart valve and inflating the balloon to plastically expand the valve member and stiffening band and expand the inner flow orifice by at least 2 mm.

The present application also discloses a method of surgically implanting a first prosthetic heart valve at a native pulmonary annulus and then implanting a second prosthetic heart valve within the first prosthetic heart valve, comprising:

providing a first prosthetic heart valve having:

a valve member with a leaflet support structure defining a plurality of upstanding commissures alternating with the same number of arcuate cusps to define an undulating outflow edge, the valve member having a plurality of flexible leaflets extending along and attached to the undulating outflow edge that coapt against one another and form a one-way valve across an inner flow orifice defining a central axis, the support structure being formed of a plastically expandable material;

a stiffening band juxtaposed against an inflow end of the valve member, the stiffening band defining a generally annular shape with an initial diameter sufficient for valve functioning and being formed of a plastically expandable material and incapable of compression smaller than the initial diameter, the stiffening band being capable of expansion of at least 2 mm in diameter; and

fabric coverings around the valve member and stiffening band, wherein the fabric coverings or sutures connecting the fabric covering supply the only coupling structure between the valve member and stiffening band;

surgically delivering and implanting the first prosthetic heart valve at the native pulmonary annulus by expanding the first prosthetic heart valve;

closing up the patient after implanting the first prosthetic heart valve at the native pulmonary annulus;

periodically monitoring a valve function of the first prosthetic heart valve; and

when the valve function diminishes below a threshold performance, advancing an expansion balloon together with a second prosthetic heart valve to a position within the first prosthetic heart valve, and inflating the balloon to plastically expand the second prosthetic heart valve outward and expand the inner flow orifice of the first prosthetic heart valve by at least 2 mm, at the same time installing the second prosthetic heart inside the first prosthetic heart valve.

Certain examples comprise a prosthetic heart valve having a cobalt-chromium alloy (or other plastically deformable metals, such as titanium alloys, stainless steels, etc..) wireform that is covered with polyester cloth. Flexible bioprosthetic leaflets are attached within the wireform in a conventional manner. The frame is smaller in size than conventional aortic valves, for example and the wire diameter used is much smaller than traditional surgical wireforms. Reducing the wire diameter and stiffness and mass will enable the frame to flex at pulmonic valve closing pressures, which are at least 5x lower than the aortic valve, increasing valve performance. Examples of leaflets of the valve are made of either porcine or bovine pericardium with thicknesses of less than about 0.35 mm (about 0.014"). The reduced mass of the leaflets and thickness will also increase valve response at low pressures.

The valve also may have an expandable stiffening band that can increase valve inflow diameter more than 8 mm with 6 atm of pressure, as with a balloon. In some cases, the stiffening band may be incrementally expanded in diameter to accommodate growth of the patient, or to accommodate a subsequently implanted percutaneous valve in a so-called valve-in-valve procedure. This feature will enable to increase the valve size percutaneously prior to replacement by at least two valve sizes; e.g., 2 mm then 4 mm, etc. An exemplary valve diameter, therefore, may be initially 13 mm, and may be expanded to 15 mm and then 19 mm at different times. The stiffener band can be as small as 10 mm ID (11 mm OD) and expandable to accommodate an about 20-mm transcatheter valve.

A further understanding of the nature and advantages will become apparent by reference to the remaining portions of the specification and drawings.

### BRIEF DESCRIPTION OF THE DRAWINGS

Features and advantages will become appreciated as the same become better understood with reference to the specification, claims, and appended drawings wherein:

FIG. 1 is an overall cutaway view of the circulatory system of the heart illustrating all four native valves;

FIG. 2 is an enlarged cutaway view of a prior art prosthetic heart valve implanted at the aortic annulus;

FIG. 3 is a perspective view of an exemplary prosthetic heart valve of the prior art;

FIGS. 4A-4D are cutaway and detail views of the prosthetic heart valve of FIG. 3 illustrating certain exemplary constructional aspects;

FIG. 5 is an enlarged cutaway view of a prosthetic heart valve of the present application implanted at the native pulmonary valve annulus;

FIG. 6 is an elevational view of a prosthetic heart valve of the present application;

FIG. 7 is an elevational view of two main structural components of the prosthetic heart valve of FIG. 6;

FIG. 8 is a perspective view of an exemplary leaflet-supporting wireform forming one of the structural components of the prosthetic heart valve of FIG. 6;

FIG. 9 is a perspective view of an exemplary expandable stiffening band forming another of the structural components of the prosthetic heart valve of FIG. 6;

FIGS. 10A-10C are elevational views of the stiffening band in different stages of expansion;

FIG. 11A is a cutaway view of the prosthetic heart valve of FIG. 6 illustrating certain exemplary constructional aspects, and FIG. 11B is an enlargement of one commissure post thereof; and

FIGS. 12A and 12B are elevational views of the prosthetic heart valve of FIG. 6 minus an outer fabric cover and sealing ring, and showing an initial state and subsequently expanded state.

### DETAILED DESCRIPTION OF CERTAIN EXAMPLES

Currently, there is a limited number to no choices for surgical pediatric valve replacement in general, and in particular for the pulmonary valve position. Most valves used for pediatric valve replacement were designed for aortic valve replacement in adults. These valves are generally too large and are designed to withstand closing pressures exceeding 120 mmHg, at least 5x pulmonic valve closing pressures. Because these valves are large and designed for higher pressures, they perform poorly when used to replace pulmonic valves in infants.

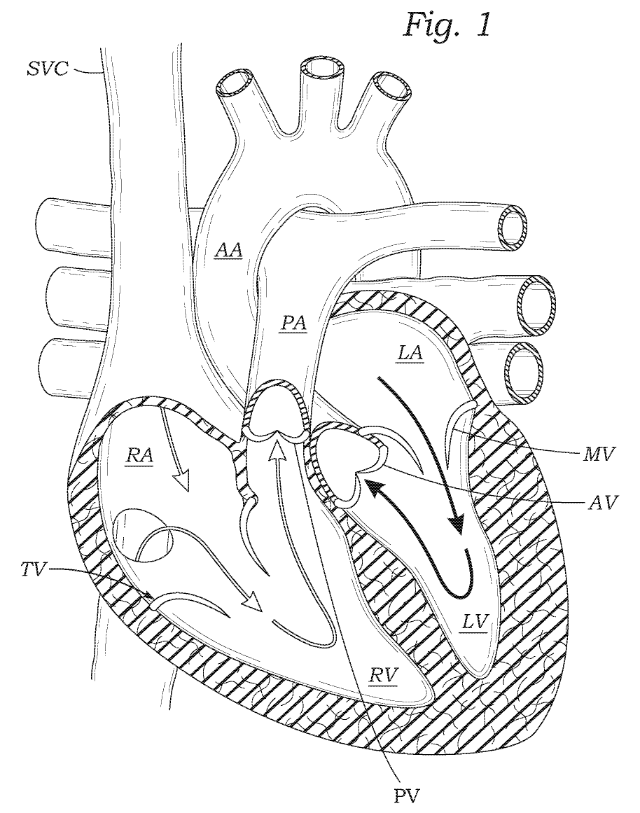

FIG. 1 is an overall cutaway view of the circulatory system of the heart in a diastolic phase illustrating all four native valves. The right ventricle RV and left ventricle LV are separated from the right atrium RA and left atrium LA, respectively, by the tricuspid valve TV and mitral valve MV; e.g., the atrioventricular valves. Additionally, the aortic valve AV separates the left ventricle LV from the ascending aorta AA and the pulmonary valve PV separates the right ventricle from the pulmonary artery PA (often called the pulmonary trunk). Each of these valves has flexible leaflets extending inward from a fibrous annulus across the respective orifices that come together or “coapt” in the flowstream to form the one-way fluid occluding surfaces. The prosthetic heart valves of the present application are primarily intended for use to replace the pulmonary valve PV, though it should be understood that the devices described herein may equally be used to replace the other valves.

The cardiac cycle involves collecting deoxygenated or venous blood on the right side of the heart and forcing it into the lungs, then porting the now oxygenated blood from the lungs through the left side of the heart and back into the circulatory system. The right atrium RA receives deoxygenated blood (shown lighter) from the venous system through the superior vena cava SVC and the inferior vena cava IVC, among other veins. During the diastolic phase, or diastole, seen in FIG. 1, the venous blood that collects in the right atrium RA is pulled through the tricuspid valve TV by expansion of the right ventricle RV. Likewise, oxygenated blood (shown lighter) is pulled through the mitral valve MV by expansion of the left ventricle LV. In the systolic phase, or systole, not shown, the heart muscle squeezes and the right ventricle RV collapses to force the venous blood through the pulmonary valve PV and pulmonary artery PA into the lungs. Similarly, the left ventricle LV collapses to force the oxygenated blood through the aortic valve AV through the ascending aorta AA and into the circulatory system. During systole, the positions of the leaflets of the four valves are opposite from what is shown in FIG. 1.

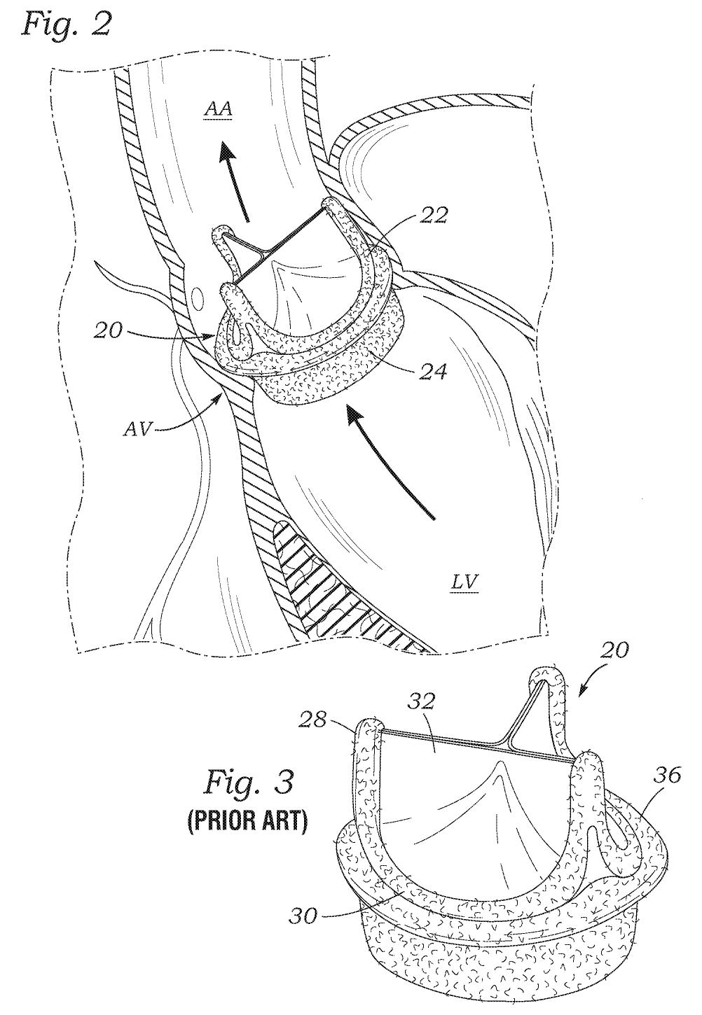

FIG. 2 is an enlarged cutaway view of a prior art prosthetic heart valve 20 implanted at the aortic annulus, and FIG. 3 is a perspective view of the exemplary bioprosthetic heart valve. The heart valve 20 is shown as a so-called “hybrid” prosthetic heart valve 20, which includes an upper valve member 22coupled to a cloth-covered anchoring skirt 24. The anchoring skirt 24 is typically a plastically-expandable metallic frame formed of struts, though it may also be self-expandable. Details of such hybrid valves are seen in more detail in US Patent Publication No. 2017/0000604, expressly incorporated herein for all purposes. Although a hybrid prosthetic heart valve is shown for comparison with the prior art, convention bioprosthetic surgical valves are similarly constructed, albeit without the anchoring skirt.

The valve member 22 of the hybrid prosthetic heart valve 20 has three upstanding commissure posts 28 projecting toward an outflow end along the flow axis alternating with three arcuate cusps 30 curving toward an inflow end. As illustrated, blood flows from the left atrium LA through the aortic valve and ascending aorta AA in an upward direction, and thus the inflow end is down and outflow end is up. Three flexible leaflets 32 are supported by the commissure posts 28 and cusps 30, and extend across a generally cylindrical flow orifice defined therewithin to form one-way occluding surfaces. Although three leaflets and three corresponding commissure posts 28 and cusps 30 are conventional, two or more than three are possible, and thus the valves described herein may have a plurality of flexible leaflets. Further, the flexible leaflets 32 are desirably bioprosthetic, such as bovine or porcine, though other biomaterials as well as synthetic materials may be used.

In the illustrated example, the heart valve 20 also includes a highly compliant sealing ring 36 extending outward therefrom at approximately the interface between the valve member 22 and the anchoring skirt 24. The sealing ring 36 may be a silicone or fabric cuff covered with fabric that helps prevent leakage around the outside of the valve once implanted. Furthermore, the sealing ring 36 is also suture-permeable and may be used to secure the valve in place in the native annulus or vessel. In the illustrated example, the native aorta valve leaflets have been resected such that the sealing ring 36 seats in a supra-annular position just above the fibrous ledge defining the aortic annulus. Alternatively, the aortic leaflets are sometimes left in place and simply compressed between the valve and the annulus.

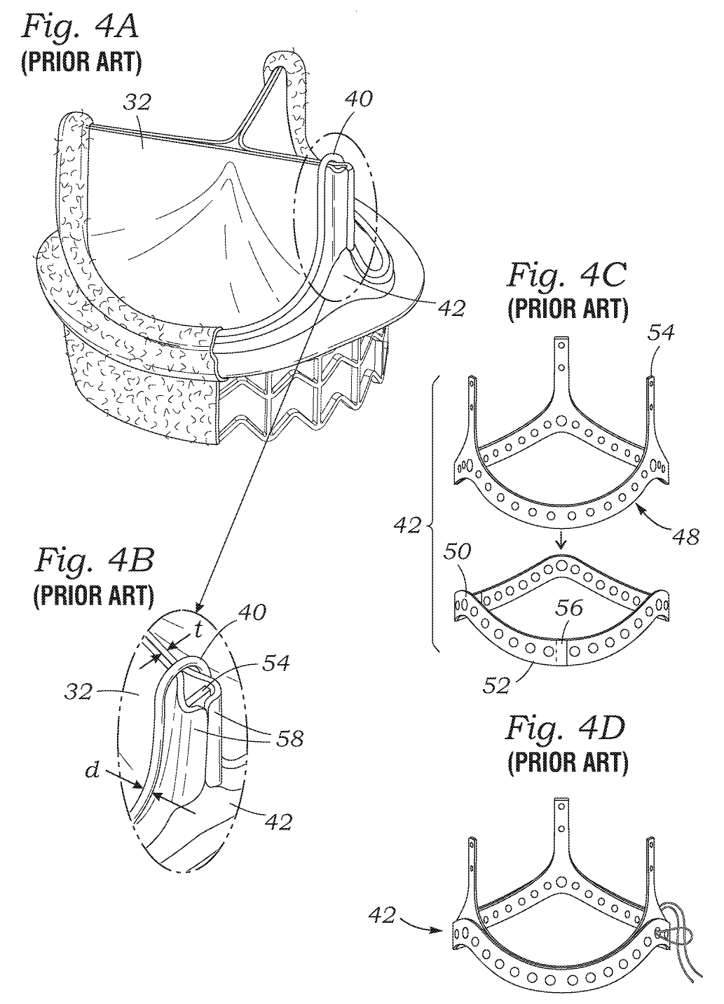

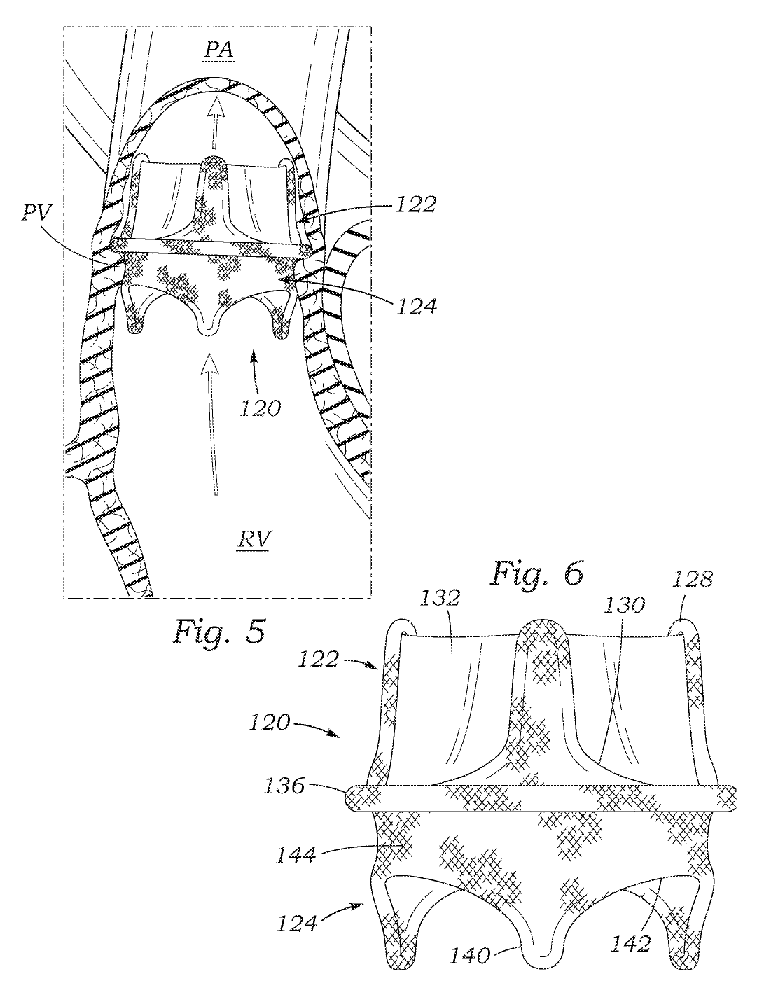

FIGS. 4A-4D are cutaway and detail views of the prosthetic heart valve 20 of FIG. 3 illustrating certain exemplary constructional aspects. The three flexible leaflets 32 are supported partly by an undulating wireform 40 as well as by a structural stent 42. The wireform 40 may be formed from a metal, such as a cobalt-chromium alloy or Nitinol, while the structural stent 42 may be metallic, plastic, or a combination of the two. The wireform 40 and structural stent 42 are conventionally covered with a polyester fabric to facilitate assembly and reduce direct blood exposure after implant.

FIGS. 4C and 4D show the exemplary inner structural stent 42 in both exploded and assembled views. The particular illustrated structural stent 42 is used in pericardial heart valves manufactured by Edwards Lifesciences of Irvine, CA. The Edwards’ Perimount® line of heart valves utilize bovine pericardial leaflets 32 with a two-part inner stent 42 comprising an assembly or composite of two concentric bands – an outer band 46 surrounding an inner band 48. The bands 46, 48 are relatively thin in radial dimension as compared to axial dimension, and both have coincident lower edges that undulate axially up and down around the circumference. The outer band 46 exhibits three truncated peaks 50 between three downwardly curved valleys or cusps 52, while the inner band 48 has generally the same shape but also extends upward at commissure posts 54. The outer band 46 in Edwards’ valves is metallic and may be formed of a strip joined by a seam 56, while the inner band 49 is a molded polymer piece so that the commissure posts 54 are highly flexible and help enable inward movement of the leaflets. Sutures may be used to couple the two bands 46, 48 together.

Other commercially-available surgical prosthetic heart valves of the prior art utilize an inner one-piece stent formed of polymer or metal and typically covered with fabric. For instance, valves sold by Medtronic, Inc. of Minneapolis, MN under the trade names Hancock I™ or Hancock II™ and Mosaic™ and Mosaic Ultra™ have polymer inner stents. Trifecta™ stented tissue valves sold by St. Jude Medical, Inc. of St. Paul, MN on the other hand have titanium stents. The inner one-piece stents again supply the main structural skeleton of the valve, and as with the Edwards’ valves comprise a thin-walled tubular member with a lower circular band that extends around the periphery of the stent, and a plurality of upstanding commissure posts. As with other conventional valves, there are three commissure posts each of which supports two adjacent leaflets. It should be understood that leaflet and supporting stent constructions other than those illustrated may be used in the prosthetic valves described herein.

A particularly useful leaflet support is seen in the Edwards’ Perimount® line of heart valves which leads to lower stresses in the pericardial leaflets 32. As seen in FIG. 4B, outer tabs 58 of adjacent leaflets 32 wrap around the flexible commissure posts 54 of the structural stent 42 at the commissures of the valve. Specifically, each leaflet 32 has an outwardly-directed tab 58 that passes underneath a U-shaped upstanding post of the wireform 40 and diverges from an adjacent tab 58 around the corresponding commissure post 54. Sutures are then used to secure the tabs 58 in this position, and the sutures may pass through holes in the commissure posts 54 seen in FIG. 4C. Other commercial valves omit the wireform and the bioprosthetic leaflets are therefore supported solely by commissure posts of an inner stent.

FIG. 4B indicates both a thickness t of the leaflets 32 and a wire diameter d for the elongated wire of the wireform 40. Conventional prosthetic surgical heart valves have overall diameters of 19–33 mm, usually in 2 mm increments. Leaflets 32 used in such valves have varying thicknesses, often due to the variability of the xenographic porcine or bovine materials used. However, for leaflets 32, made from more uniform thickness sheets of bovine pericardium, the thickness t typically ranges between 0.014–0.023 inches. Wireforms 40 may have a wire diameter d of between 0.020–0.026 inches. These dimensions are suitable for heart valves used at the aortic or mitral positions, where the blood flow pressures are substantial, but not for the pulmonary position where the pressures are orders of magnitude less. The relatively bulky and stiff surgical valves of the prior art, even the smallest diameter versions, tend to perform poorly as pulmonary valve replacements.

FIG. 5 is an enlarged cutaway view of a prosthetic heart valve 120 of the present application implanted at the position of the native pulmonary valve. Aspects of the prosthetic heart valve 120, in particular its overall size and the size of certain components, are particularly useful at the native pulmonary annulus, though the valve could be utilized at other native annuluses. The heart valve 120 is configured like a “hybrid” prosthetic heart valve 120, and has an upper valve member 122 coupled to a cloth-covered stiffening band 124 juxtaposed against an inflow end of the valve member. The stiffening band 124 is typically a plastically-expandable metallic frame formed of struts. If a plastically-expandable metallic frame is used, the stiffening band 124 may be expanded by inflating a balloon therewithin. Preferably, the balloon inserts all the way into the flow orifice of the heart valve 120 and radially expands both the valve member 122 and the stiffener band 124 in equal proportions.

As seen in FIG. 6, the valve member 122 has three upstanding commissure posts 128 projecting toward an outflow end along the flow axis alternating with three arcuate cusps 130 curving toward an inflow end. As illustrated, blood flows from the right ventricle RV through the pulmonary valve PV and pulmonary artery PA in an upward direction, and thus the inflow end is down and outflow end is up. Three flexible leaflets 132 are supported by the commissure posts 128 and cusps 130, and extend across a generally cylindrical flow orifice defined therewithin to form one-way occluding surfaces. Although three leaflets and three corresponding commissure posts 128 and cusps 130 are conventional, two or more than three are possible, and thus the valves described herein may have a plurality of flexible leaflets. Further, the flexible leaflets 132 are desirably bioprosthetic, such as bovine or porcine, though other biomaterials as well as synthetic materials may be used.

In the illustrated example, the heart valve 120 also includes a highly compliant sealing ring 136 extending outward therefrom at approximately the interface between the valve member 122 and the stiffening band 124. The sealing ring 136 may be a silicone or fabric cuff covered with fabric that helps prevent leakage around the outside of the valve once implanted. Furthermore, the sealing ring 136 is also suture-permeable and may be used to secure the valve in place in the native annulus or vessel. In the illustrated example, the native pulmonary valve leaflets have been resected such that the sealing ring 136 seats in a supra-annular position just above the fibrous ledge defining the pulmonary annulus. Alternatively, the leaflets are sometimes left in place and simply compressed between the valve and the annulus.

The illustrated stiffening band 124 has a plurality, preferably three, of downwardly-projecting commissure posts 140 alternating with a plurality of upwardly-curved cusp edges. These shapes are formed by an inner member that defines the stiffening band 124, as will be described in more detail below, covered in a fabric 144. The fabric covering 144 may surround just the inner member, or maybe common to both the sealing ring 136 and the inner member.

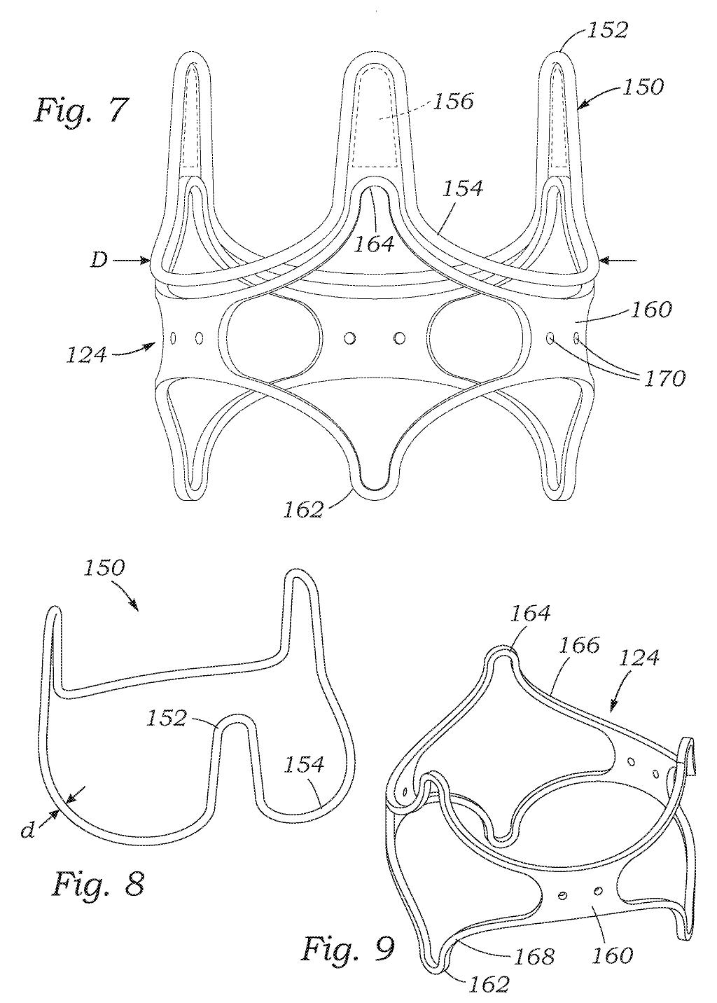

FIG. 7 is an elevational view of two main structural components of the prosthetic heart valve of FIG. 6; namely, FIG. 8 shows an exemplary leaflet-supporting wireform 150 and FIG. 9 shows the inner member that defines the expandable stiffening band 124 juxtaposed against an inflow end of the wireform. The wireform 150 has an exaggerated undulating shape with a plurality (e.g., three) upstanding commissures 152 alternating with the same number of arcuate cusps 154. The wireform 150 defines an outflow end of the heart valve 120, wherein the commissures 152 extend toward an outflow end and the cusps 154 curve toward an inflow end of the valve. The wireform 150 is constructed of an elongated, continuous wire-like element such that both the commissures 152 and cusps 154 defined U-shaped portions, with the commissures being significantly narrower and taller in an axial dimension than the cusps. When rotated about a central axis, the wireform 150 defines a tubular or slightly conical surface of revolution.

The wireform 150 represents an annular support within the valve member 122 which provides an undulating structural shape around which the flexible leaflets 132 are affixed. This shape enables the leaflets 132 to function as one-way fluid occluding surfaces within the flow orifice defined by the valve member 122. As mentioned above, prior art prosthetic valves such as those available from Edwards Lifesciences, Medtronic, Inc., and St. Jude Medical, Inc. have varying styles of inner leaflet support members, and the wireform 150 may be replaced by a number of different structures. One advantageous aspect, however, is that whatever annular leaflet support is utilized, it is flexible along with the stiffening band 124 to enable radial expansion. For instance, U.S. Patent No. 10,543,085, expressly incorporated herein, describes a number of such valve support members which are expandable to facilitate valve-in-valve procedures. In addition to enabling a valve-in-valve procedure, the expandability of the heart valve 120 provides the ability to expand the valve post-implant in stages to accommodate anatomical growth of the patient. This becomes more important when the patient is initially an infant or child, and growth is inevitable. More about such incremental valve expansion will be provided below.

FIG. 7 also illustrates in phantom a number of small inserts 156 that may be used to secure the valve leaflets 132. The inserts 156 are shown in phantom to emphasize that they provide just one means for leaflet 132 securement. As with the flexible commissure posts 54 on the inner band 48 of the stent 42 seen in FIG. 4D, the leaflets 132 may be provided with outwardly-directed tabs like the tabs 58 in FIG. 4B. The leaflet tabs may be inserted under the U-shaped commissures 152 and wrapped around and secured to the inserts 156. Because the inserts 156 are disconnected elements, however, they do not add annular stiffness to the valve 120.

One problem associated with the pediatric heart valve replacement surgery is that when a valve is implanted into children or adolescents, the subsequent growth of the patient may render the heart valve too small for its intended function, thus abnormally constricting the annulus or vessel. Follow-up surgery would be necessary to replace the originally implanted heart valve with a larger one suitable for the then-current size of the patient. Consequently, a beneficial aspect of the heart valves 120 described herein is that they are expandable.

The stiffening band 124 has an annular shape which, when rotated, defines a tubular surface of revolution. The stiffening band 124 comprises an alternating series of solid plate segments and expandable segments around its periphery. Namely, the band 124 has a series of plate segments 160 connected by expandable struts or wire-like elements. In the illustrated example, each expandable segment has an inflow peak 162 opposite an outflow peak 164 connected to adjacent plate segments 160 via arcuate struts 166, 168, respectively. The combination of the peaks 162, 164 and the struts 166, 168 form a somewhat diamond-shaped arrangement of struts in between the plate segments 160. Apertures 170 through each of the plate segments 160 may be provided to facilitate attachment of the outer fabric covering 144 (FIG. 6).

The plate segments 160 create nodes or regions where the stiffening band 124 cannot expand, with the expansion only occurring within the segments having the expandable peaks 162, 164 and struts 166, 168. Preferably, the expansion segments are circumferentially centered or aligned with the U-shaped commissures 152 of the wireform 150 so that upon expansion of the stiffening band 124 the commissures 152 spread apart evenly. Conversely, the cusps 154 of the wireform 150 are centered or aligned with the plate segments 160 and are thus not subject to expansion forces. The leaflets 132 are configured to function over a range of diameters of the wireform 150, such as providing slightly larger or loose leaflets for the initial valve size which then can accommodate some expansion thereof and still coapt with each other. For instance, an initial valve size may be incrementally increased by 2 mm, and then again by another 2mm over time, and the leaflets 132 still function to provide one-way blood flow occluding surfaces. An example is an initial size of 13 mm (the “size” being the labeled valve size or diameter corresponding to a measured flow orifice diameter) which may be increased over time to 15 mm and then eventually to 19 mm.

The stiffening band 124 coupled to valve member 122 in the overall valve 120 can be as small at 10 mm ID (11 mm OD) and expandable to accommodate a 20 mm transcatheter valve. Depending on the design of the leaflet 132 there may be a practical limit to the range of expansion, and a complete prosthetic valve resection and replacement may be necessary for some patients. Alternatively, since the valve 120 is expandable, a subsequent valve-in-valve procedure may be indicated.

In the illustrate example, the plate segments 160 extend around the circumference of the stiffening band 124 a lesser amount than the intervening expansion segments. For example, each plate segment 160 may extend around a span of about 30° while each expansion segment comprising the peaks 162, 164 and struts 166, 168 extends around about 90°. In general, the angular span of each plate segment 160 is between about 17–75% of the span of one of the expansion segments, and in absolute terms is between about 15–50° of the entire circumference. This balance enables significant expansion while providing enough stiffness to the band 124 to maintain a particular diameter against physiological forces associated with pulsatile blood cycling and attendant orifice flexing. As mentioned, the peaks 162, 164 and struts 166, 168 desirably form a diamond-like shape below each U-shaped commissure 152 of the wireform 150 with the upper or outflow peaks 164 extending up within the wire-like commissure 152. Indeed, the varying curvature of the arcuate peaks 164 preferably mimics that of the U-shaped commissure 152. The lower or inflow peaks 162 are preferably a mirror image of the upper peaks 164. Of course, as mentioned, the expansion segments may be differently configured, such as having multiple rows of expandable struts and the like as with traditional transcatheter valve stents.

As mentioned above, the structural components within the valve member 122 are expandable, even though they may incorporate an annular member such as the wireform 150. Significantly, the valve 122 omits any stiff annular members, such as the inner stent 42 shown in FIG. 4D for the Edwards pericardial valve. The wireform 150 is capable of expansion and may be made of a plastically-expandable material such as cobalt-chromium alloy such as Elgiloy® alloy (or other plastically deformable metals, such as titanium alloys, stainless steels, etc.). The wireform 150 may alternatively be formed from a flexible material, such as a flexible metal like Nitinol or a polymer, in which case the wireform expands with the plastically-expandable band 124. The inner member of the stiffening band 124 is formed of a continuous band of plastically-expandable material, such as stainless steel, a titanium alloy or a cobalt-chromium alloy. Because of the expandable segments in the stiffening band 124, it may be expanded to a number of different sizes.

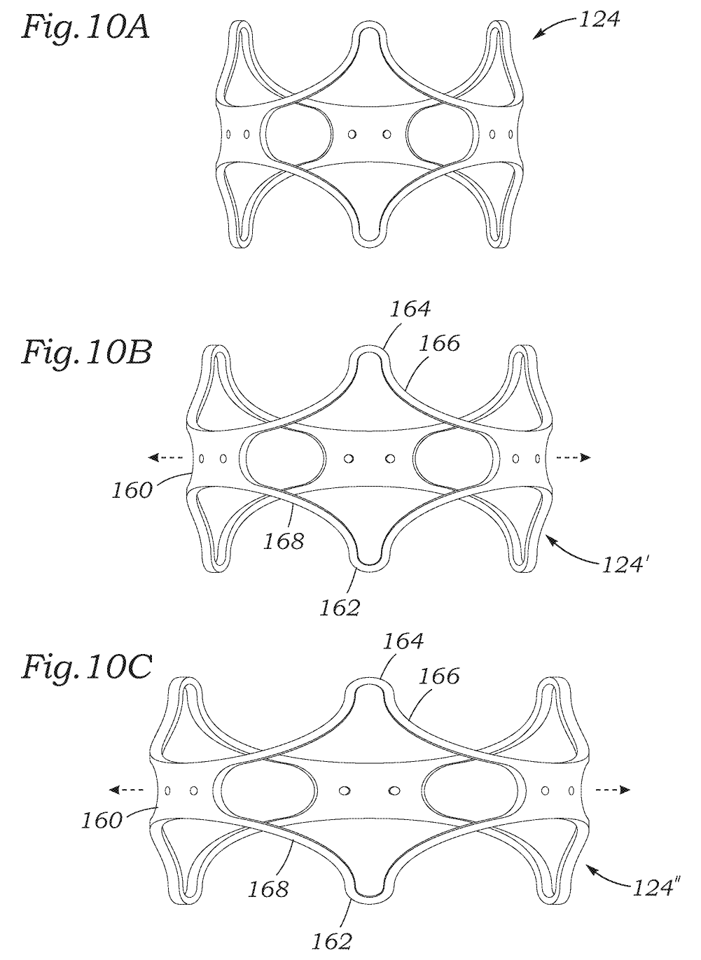

FIGS. 10A-10C are elevational views of the stiffening band 124 in different stages of expansion. FIG. 10A shows the stiffening band 124 prior to any expansion. FIG. 10B indicates outward expansion to define a modified stiffening band 124'. And finally, FIG. 10C indicates further outward expansion to define another modified stiffening band 124''. At each stage of expansion, the U-shaped peaks 162, 164 and connective struts 166, 168 straighten out somewhat. By distributing the expandable segments around the stiffening band 124, and in particular in regions corresponding to the commissure posts of the valve member 122, the heart valve 120 maintains circularity and functionality.

It is important to understand that the prosthetic heart valve 120 of the present application is configured for surgical delivery, as opposed to transcatheter valves which are compressed into a small profile and delivered though a catheter or other access tube. That is, the stiffening band 124 is formed of a plastically-expandable material which is capable of expansion but not compression from its initial shape, such as shown in FIG. 10A. Of course, though excessive force may indeed compress the stiffening band 124 from the size of FIG. 10A, the band will crumple and be non-functional as a result. Thus, the term “adapted for surgical delivery” refers to heart valves that cannot be compressed to pass through a tube, and must be delivered with the patient on cardiopulmonary bypass.

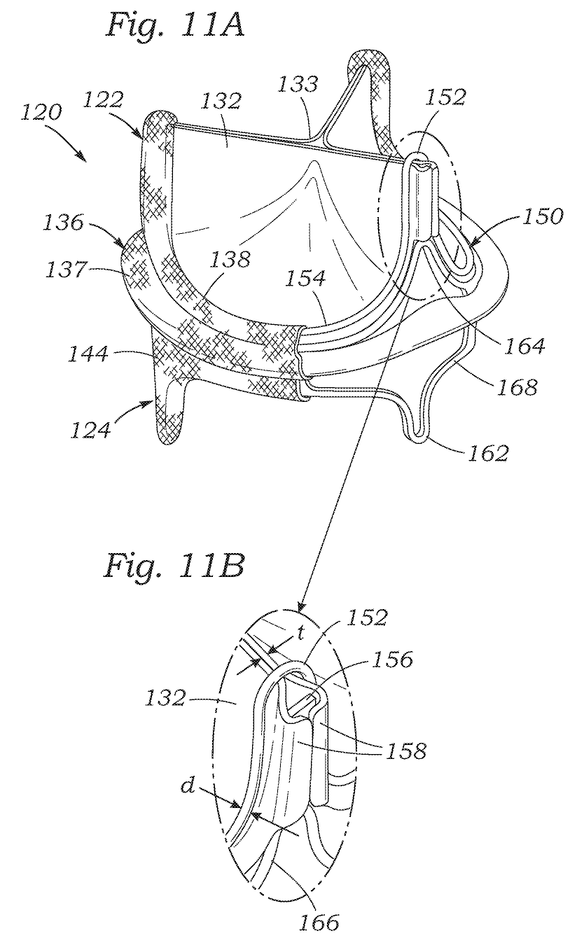

FIG. 11A is a cutaway view of the prosthetic heart valve 120 of FIG. 6 showing one side without the outer fabric cover 144, and FIG. 11B is an enlargement of one commissure post thereof. As with the earlier described surgical heart valves, the heart valve 120 incorporates bovine pericardium in three separate leaflets 132 that are supported by the three commissures 152 and cusps 154 of an undulating wireform 150. The leaflets 132 each have a somewhat semi-circular shape with outwardly-extending tabs 158 (FIG. 11B) at opposite ends of an upper free edge 133. An arcuate cusp edge of each of the leaflets 132 secures around the arcuate cusps 154 using sutures through a fabric cover 138 around the wireform 150. The tabs 158 of adjacent leaflets 132 extend underneath one of the wireform commissures 152 and wraps around the insert 156. Sutures (not shown) are used to secure the tabs 156 to the insert 156.

FIG. 11A shows the location of the sealing ring 136, radially outward of the junction between the valve member 122 and stiffening band 124. The sealing ring 136 may undulate slightly to match the undulating shapes of the wireform 150 and upper edge of the stiffening band 124. The sealing ring 136 also has a fabric cover 137, which may be common with either the valve member cover 138 or the stiffening band cover 144, or the three covers may be separate pieces. In any event, the fabric covers 137, 138, 144 themselves or sutures between separate covers make up the structural connection between the valve member 122 and stiffening band 124. That is, FIG. 7 shows the wireform 150 above and separated structurally from the stiffening band 124, and there are no other structural (e.g., non-fabric) components therebetween. This flexible assembly facilitates the ability of the valve member 122 to flex in and out, and also helps render the entire valve 120 more flexible for when expansion to a larger diameter is needed.

FIG. 11B indicates a thickness t of one of the leaflets 132. For the pulmonary position, the overall diameter of the valve 120 is decreased from typical aortic or mitral valves, and the leaflets 132 are reduced in thickness also, to enable opening and closing from the lower fluid pressure pulses. Pulmonic valve closing pressures of between 4–6 atm are at least 5x lower than those experienced at the aortic valve, for example. As mentioned above, typical leaflet thickness for aortic or mitral valves ranges between about 0.014‒0.023 inches (about 0.36‒0.58 mm). In contrast, the thickness t of one of the leaflets 132 for a pulmonary valve 120 is less than 0.014", for instance between about 0.004‒0.013 inches (about 0.1‒0.33 mm). Likewise, the wireform 150 has a smaller wire diameter d of between about 0.009‒0.019 inches (about 0.23‒0.48 mm), whereas the diameter of a wireform for the larger aortic or mitral valves is between about 0.02‒0.026 inches (about 0.5‒0.66 mm).

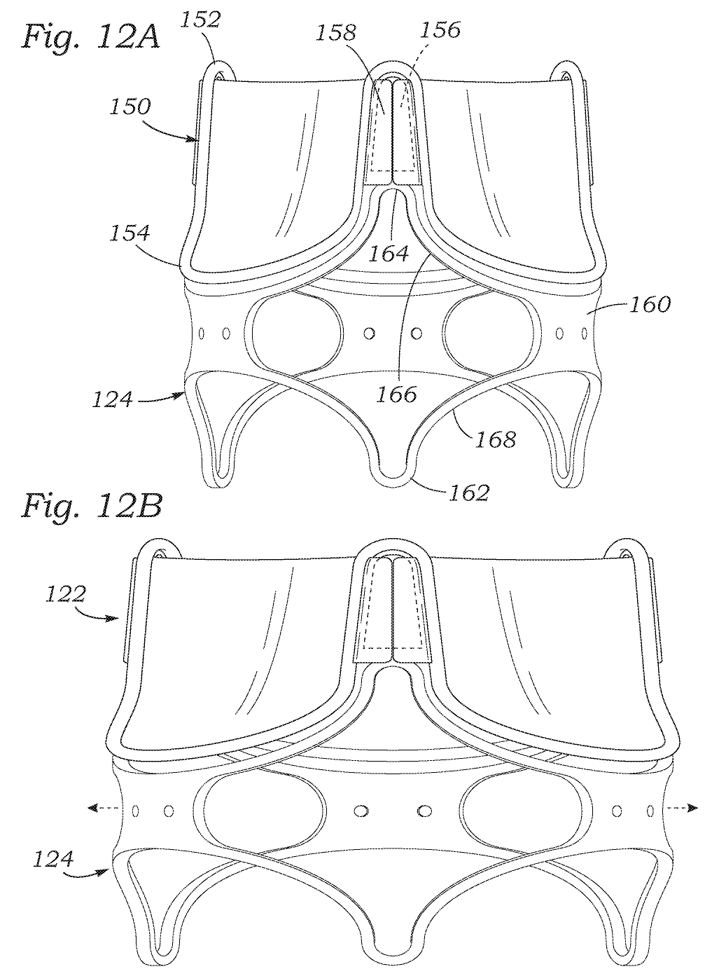

FIGS. 12A and 12B are elevational views of the prosthetic heart valve 120 of FIG. 6 minus outer fabric covers 137, 138, 144 and sealing ring 136 showing an initial state and subsequent expanded state. The initial state may have an overall diameter of 13 mm, while the expanded state may be 15 mm, or after a second expansion, 19 mm. Smaller pulmonary valves are contemplated, down to an initial state with a diameter of 11 mm. Due to the coupling of fabric covers 137, 138, 144, the valve member 122 expands along with the stiffening band 124, such as by inflating a balloon within the stiffening band 124.

A method of surgical implanting a prosthetic heart valve at a native pulmonary annulus and enlarging the size of the implanted prosthetic heart valve is contemplated. The method involves procuring and preparing (e.g., providing) any of the prosthetic heart valves described herein. The prosthetic heart valve is surgically delivered and implanted at the native pulmonary annulus by expanding the prosthetic heart valve, after which the patient is closed up. Periodically, a size of the native pulmonary annulus or an analog of the size is measured. An analog of the size may be the size of one of the other native annuluses, which often grow in proportion to the pulmonary annulus. When the size or analog reaches a threshold magnitude, such as when the size or analog grows by 2 mm or more, the prosthetic heart valve is expanded. For instance, an expansion balloon is advanced within the prosthetic heart valve and the balloon inflated to plastically expand the valve member and stiffening band and expand the inner flow orifice by at least 2 mm.

A method of surgical implanting a first prosthetic heart valve at a native pulmonary annulus and then implanting a second prosthetic heart valve within the first prosthetic heart valve is also contemplated. The method involves procuring and preparing (e.g., providing) any of the prosthetic heart valves described herein. The prosthetic heart valve (first valve) is surgically delivered and implanted at the native pulmonary annulus by expanding the prosthetic heart valve, after which the patient is closed up. Periodically, a valve function of the first prosthetic heart valve is monitored, such as by echocardiography. If and when the valve function diminishes below a threshold performance, such as when regurgitation is detected, a second valve in a valve-in-valve procedure is implanted. The valve-in-valve procedure commences by advancing an expansion balloon together with a second prosthetic heart valve to a position within the first prosthetic heart valve, and inflating the balloon to plastically expand the second prosthetic heart valve outward. This expands the inner flow orifice of the first prosthetic heart valve by at least 2 mm, at the same time installs the second prosthetic heart inside the first prosthetic heart valve.

While the foregoing is a complete description of the preferred examples, various alternatives, modifications, and equivalents may be used. Moreover, it will be obvious that certain other modifications may be practiced within the scope of the appended claims.

What is claimed is:

1. A prosthetic surgical heart valve adapted for surgical delivery and implant at a native annulus, comprising:
a valve member with a leaflet support structure defining a plurality of upstanding commissures alternating with the same number of arcuate cusps to define an undulating outflow edge, the valve member having a plurality of flexible leaflets extending along and attached to the undulating outflow edge that coapt against one another and form a one-way valve across an inner flow orifice defining a central axis, the support structure being formed of a plastically expandable material;
a stiffening band comprising an alternating series of solid plate segments and expandable segments around a periphery thereof juxtaposed against an inflow end of the valve member, the stiffening band defining a generally annular shape with an initial diameter sufficient for valve functioning, the stiffening band being formed of a plastically expandable material and being incapable of compression smaller than the initial diameter, the stiffening band being capable of expansion of at least 2 mm in diameter; and
fabric coverings around the valve member and stiffening band, wherein the fabric coverings and/or sutures connecting the fabric covering to the valve member and stiffening band supply the only coupling structure between the valve member and stiffening band and there are no other non-fabric components therebetween.

2. The heart valve of claim 1, wherein the expandable segments comprise struts or wire-like elements.

3. The heart valve of claim 1, wherein each expandable segment has an inflow peak opposite an outflow peak connected to adjacent plate segments via arcuate struts.

4. The heart valve of claim 3, wherein the inflow and outflow peaks and struts form a diamond-shaped arrangement of struts in between the plate segments.

5. The heart valve of claim 1, wherein each of the plate segments has a plurality of apertures therethrough to facilitate attachment of the fabric covering.

6. The heart valve of claim 1, wherein both the valve member and stiffening band are formed of a cobalt-chromium alloy.

7. The heart valve of claim 1, further including a sealing ring attached radially outward of a junction between the valve member and stiffening band, the sealing ring having a fabric covering.

8. A prosthetic surgical heart valve adapted for surgical delivery and implant at a native annulus, comprising:
a valve member with a leaflet support structure defining a plurality of upstanding commissures alternating with the same number of arcuate cusps to define an undulating outflow edge, the valve member having a plurality of flexible leaflets extending along and attached to the undulating outflow edge that coapt against one another and form a one-way valve across an inner flow orifice defining a central axis, the support structure being formed of a plastically expandable material, and the leaflets each having a thickness t of between about 0.004–0.013 inches (0.1–0.33 mm);
a stiffening band comprising an alternating series of solid plate segments and expandable segments around a periphery thereof juxtaposed against and coupled to an inflow end of the valve member, the stiffening band defining a generally annular shape with an initial diameter sufficient for valve functioning, the stiffening band being formed of a plastically expandable material and being incapable of compression smaller than the initial diameter, the stiffening band being capable of expansion of at least 2 mm in diameter; and
fabric coverings around the valve member and stiffening band.

9. The heart valve of claim 8, wherein the expandable segments comprise struts or wire-like elements.

10. The heart valve of claim 9, wherein each expandable segment has an inflow peak opposite an outflow peak connected to adjacent plate segments via arcuate struts.

11. The heart valve of claim 10, wherein the inflow and outflow peaks and struts form a diamond-shaped arrangement of struts in between the plate segments.

12. The heart valve of claim 8, wherein each of the plate segments has a plurality of apertures therethrough to facilitate attachment of the fabric covering.

13. The heart valve of claim 8, wherein both the valve member and stiffening band are formed of a cobalt-chromium alloy.

14. The heart valve of claim 8, further including a sealing ring attached radially outward of a junction between the valve member and stiffening band, the sealing ring having a fabric covering.

15. A prosthetic heart valve surgical adapted for surgical delivery and implant at a native annulus, comprising:
a valve member with a leaflet support wireform defining a plurality of upstanding commissures alternating with the same number of arcuate cusps to define an undulating outflow edge, the wireform having a wire diameter d of between about 0.009‒0.019 inches (about 0.23‒0.48 mm), the valve member having a plurality of flexible leaflets extending along and attached to the undulating outflow edge that coapt against one another and form a one-way valve across an inner flow orifice defining a central axis, the wireform being formed of an expandable material;
a stiffening band comprising an alternating series of solid plate segments and expandable segments around a periphery thereof juxtaposed against and coupled to an inflow end of the valve member, the stiffening band defining a generally annular shape with an initial diameter sufficient for valve functioning, the stiffening band being formed of a plastically expandable material and being incapable of compression smaller than the initial diameter, the stiffening band being capable of expansion of at least 2 mm in diameter; and
fabric coverings around the valve member and stiffening band.

16. The heart valve of claim 15, wherein the expandable segments comprise struts or wire-like elements.

17. The heart valve of claim 15, wherein each expandable segment has an inflow peak opposite an outflow peak connected to adjacent plate segments via arcuate struts.

18. The heart valve of claim 17, wherein the inflow and outflow peaks and struts form a diamond-shaped arrangement of struts in between the plate segments.

19. The heart valve of claim 15, wherein each of the plate segments has a plurality of apertures therethrough to facilitate attachment of the fabric covering.

20. The heart valve of claim 15, wherein both the valve member and stiffening band are formed of a cobalt-chromium alloy.

21. The heart valve of claim 15, further including a sealing ring attached radially outward of a junction between the valve member and stiffening band, the sealing ring having a fabric covering.
